(19) United States
(12) Patent Application Publication
(10) Pub. No.: US 2010/0294287 A1
(43) Pub. Date: Nov. 25, 2010

(54) Treatment of Chronic or Recurrent Cellulitis of the Lower Limb

(76) Inventors: Seshadri Raju, Jackson, MS (US); Peter Neglen, Brandon, MS (US)

Correspondence Address: JONES, WALKER, WAECHTER, POITEVENT, CARRERE;& DENEGRE, L.L.P., 5TH FLOOR, FOUR UNITED PLAZA, 8555 UNITED PLAZA BOULEVARD, BATON ROUGE, LA, 70809, US

(21) Appl. No.: 12/470,695

(22) Filed: May 22, 2009

(51) Int. Cl. A61B 17/00

(52) U.S. Cl. 128/898

(57) ABSTRACT

Recurrent or chronic cellulitis is diagnosed, and the ouflow areas are examined for stenosis or obstructive lesions, and stented as needed.

### FIELD OF INVENTION

Diagnoisis and treatment of recurrent or chronic cellulitis with stents

### BACKGROUND OF THE INVENTION

Cellulitis is a diffuse infection of connective tissue with severe inflammation of dermal and subcutaneous layers of the skin. Cellulitis can be caused by normal skin flora or by exogenous bacteria, and often occurs where the skin has previously been broken: cracks in the skin, cuts, blisters, burns, insect bites, or surgical wounds. Cellulitis can accompany traumatic wound, such as compound bone fractures, shot gun wounds etc, and can also accompany non-venous skin ulcers, such as fungus infections and diabetes. Celluitis is normally accompanied with redness and pain and swelling of the infected area, and is generally treated with antibiotics with gram positive coverage. Cellulitis generally occurs in patients in the presence of epithelial breakdown—dermatitis and ulceration. However, cellulitis can occur in the absence of such skin changes. Limb swelling is commonly present with cellulitis, even when ulceration and dermatitis is lacking. The term spontaneous cellulitis is used herein to characterize cellulitis in the absence of ulceration and dermatitis, to emphasize the absence of an obvious entry point for the infection such as an ulcer, eczema, traumatic skin abrasion or interdigital skin fissure. It is generally assumed that bacterial invasion occurs through invisible micro abrasions or passage through intact skin or appendages with compromised dermal barrier, even though embolic seeding cannot be excluded. Systemic signs such as fever, leucocytosis, and left shift are not frequently present, even though local signs of inflammation are unmistakable (spreading erythema, increased swelling and pain). Blood cultures are seldom positive. The problem can become recurrent, meaning two or more episodes of the inflammation occur over a period of a year, or three or more episodes over a five year period, or five or more episodes over any time period

Chronic venous disease (CVD, sometimes referred to as chronic venous insufficiency) is a condition of the venous system. Patients with severe symptoms of CVD exhibit one or more following features: severe pain, swelling, stasis dermatitis and venous ulcer. Causes of CVD are generally attributable to obstruction (also called stenosis) or reflux (a malfunctioning valve) or a combination. If a thrombotic or non-thrombotic vein lesion(s) causing venous obstruction or stenosis is diagnosed using the existing testing protocols (such as pressure measurements, duplex scans, venography and CT/MR imaging), then intravascular imaging techniques, such as intravascular ultrasound (IVUS) or venography can be used to determine the exact obstruction location and extent; percutaneous balloon angioplasty and/or stent placement is used to treat the obstruction at the same time.

Until very recently, reflux was thought to be the main pathological mechanism for CVD. CVD is now recognized to be largely due to post-thrombotic and non-thrombotic etiologies occurring roughly in equal proportion in either variety. Outflow obstruction (a stenosis in the outflow veins—the iliac vein, and the adjacent vena cava and femoral vein segments) has been established as a common component of post-thrombotic disease, and in a majority of cases, ²⁶ an iliac vein compressive/obstructive lesion has been found. Non-thrombotic iliac vein lesions (NIVL) have been found in over 90% of ‘primary’ CVD cases examined by the inventors. ²⁸ These asymptomatic lesions are thought to be a permissive lesion that remains silent until additional injury, infection or venous pathology (reflux) renders the limb symptomatic.

Cellulitis of the lower limbs (foot, ankle, calf and thigh areas) may also be present with CVD. In this instances, cellulitis commonly occurs in the presence of epithelial breakdown. In a small number of CVD cases, (estimated at about 3-5%), spontaneous cellulitis occurs. Limb swelling is often but not always present in such cases. Infrequently, when cellulitis is not active, the limb appears normal and asymptomatic. If the cellulitis is either recurrent, or chronic (chronic means that symptoms of cellulitis persist after antibiotic treatment), long term relief by antibiotics alone is insufficient.

### SUMMARY OF THE INVENTION

In the course of treating obstructive venous disease with stent placement, the inventors have found that recurrent cellulitis or chronic cellulitis (either spontaneous or non-spontaneous) are indicators of outflow obstructions in the common and external iliac veins, and in some cases, in the adjoining caval and femoral segments. Correction of the outflow obstruction by stent placement is often curative. Hence, the inventors have found that the presence of recurrent or chronic cellulitis, with or without the presence of swelling or pain or other indicators of CVD, is an indicator of outflow obstruction. In this instance, examination of the outflow area for stenosis and stenting of any stenosis found is corrective in a majority of cases. Correction of venous outflow obstruction in the outflow areas afforded relief from recurrent or chronic cellulitis in the majority of cases studied by the inventors.

### PREFERRED EMBODIMENT OF THE INVENTION

The invention is a method of treating chronic or recurrent cellulitis of the lower limbs. The method involves diagnosing chronic or recurrent cellulitis in the lower limbs, examining the outflow areas for stenosis, and stenting any stenosis found. Physicians can diagnose cellulitis of the lower limb by a physical inspection of the limb, looking for the presence of redness and swelling, with or without entry points for bacterial infection. When examining a patient for cellulitis, the venous clinical severity scoring system (“CEAP”)(see Beebe H G, Bergan J J, Bergqvist D et al. Classification and grading of chronic venous disease in the lower limbs. A consensus statement. Eur J Vasc Endovasc Surg 1996; 12(4):487-91; discussion 491-2, hereby incorporated by reference) is preferred as the template in clinical assessment. Using the CEAP method of classifying CVD, limbs having C0 (normal), C1 (spiders); C2 (varices); and C3 (swelling) are considered in spontaneous cellulitis—C4, C5, and C6, which involve skin breakdown of various types are considered as exhibiting non- spontaneous cellulitis. A detailed venous history included details of cellulitis such as number of episodes, first and last attack dates. Limb pain is preferred to be scored on visual analogue scale of 0 to 10 (“VAS”)(see Rutherford R B, Padberg F T Jr, Comerota A J, Kistner R L, Meissner M H, Moneta G L, Venous severity scoring: An adjunct to venous outcome assessment. J Vasc Surg 2000; 31(6):1307-12; and see also Scott J, Huskisson E C. Graphic representation of pain. Pain 1976; 2(2):175-84, both hereby incorporated by reference), with limb edema scored from 0 to 3 (Gr. 0—none; Gr. 1—pitting; Gr. 2 ankledema; Gr. 3—gross). Patients with an obvious entry point of infection through skin breakdown from a traumatic wound, such as compound bone fractures, shot gun wounds etc, and non-venous skin ulcers, such as fungus infections and diabetes induced infection are excluded from this method of diagnosis and stent treatment of venous outflow obstruction. Also excluded are other conditions that may resemble cellulitis in certain clinical features, such as allergic dermatis, contact dermatis, inflammatory changes associated with collagen diseases, such as scleroderma and rheumatic disease. Also excluded are numerous dermatological and autoimuune diseases that may exhibit an inflammation without an infective basis. Diagnsotic tests are available to distinguish these types of diseases from cellulitis.

This invention only applies to patients with spontaneous or non-spontaneous cellulitis with or without other symptoms and signs of venous disease such as swelling, stasis skin dermatitis, hyperpigmentation, lipodermatosclerosis or active/healed venous ulcer.

Once recurrent or chronic cellulitis is determined, outflow obstruction or stenosis is indicated in the common and external iliac veins and the adjoining caval and femoral segments. A single episode of spontaneous cellulitis may or may not be an indicator of iliac vein outflow obstruction, but as the remedy of outflow obstruction is an invasive procedure (stenting), at least two episodes of acute cellulits or continuing chronic cellulitis are preferred to trigger the diagnosis of outflow obstruction. Once the diagnosis of recurrent or chronic sellulitis is made, the outflow veins are investigated for stenosis.

Stenosis may be indicated by undertaking a detailed set of venous investigations including duplex, ambulatory venous pressure measurement, such as air plethysmography, and arm/foot pressure measurement. An isotope lymphangiogram may be obtained, as well as other contrast studies included ascending and transfemoral venography with femoral exercise pressure measurement. See Raju S, Neglen P. High prevalence of nonthrombotic iliac vein lesions in chronic venous disease: a permissive role in pathogenicity. J Vasc Surg 2006; 44(1):136-43; discussion 144, hereby incorporated by reference. However, venous laboratory parameters are only about 50% sensitive for venous obstruction. It is preferred that the outflow areas (primary candidate being the lilac vein) be examined using IVUS (see A Method for Diagnosing and Treating CVD—Ser. No. 11/753,295 Filed May 24, 2007, hereby incorporated by reference). IVUS is an invasive technique (an ultrasonic probe and receiver is placed in the venous system) where the interior of the vein is visualized by ultrasound. IVUS typically provides more detail of the obstructive lesion than venography, such as measuring the cross sectional area of a vein, and often the true extent of the lesion or stenosis per IVUS is more severe than suggested by venography.

The common and external iliac veins are the primary candidates, with the adjoining caval and femoral segments being secondary candidate areas to search for outflow restrictions. In general, stenotic lesions that show an obstruction representing about a 50-60% or greater reduction in lumen cross-sectional area are identified in the candidate areas are to be corrected with stents. The degree or percentage of narrowing is based upon the lumen size of adjacent “unobstructed” vein segments. Hence, stenting is indicated with changes in lumen diameter from the expected lumen diameter, as determined from adjacent areas that are not obstructed. Preferably, the degree of obstruction is based upon IVUS measurements, which can accurately measure lumen diameter and cross-section area. It is preferred to measure lumen size as a cross section area, as opposed to a diameter, for with an obstructed lumen, the actual shape may present more than one “diameter”—such as in an oblong lumen having a major diameter and a minor smaller diameter. If diameter sized is used (for instance, if IVUS is not the imaging technique) the smallest diameter is preferred, (or an average of the diameters could be used).

To perform the stenting operation, for instance, a mid-thigh ipsilateral antegrade femoral vein access may be made under ultrasound guidance with use of a sealant device (such a Vasoseal) employed at the entry point post-procedure. A guidewire is inserted and moved to a target site. A balloon tipped catheter is then advanced over the guidewire to the target site, and dilated using suitable high pressure, such as 16 atm. The balloon is then deflated, and removed (this procedure is often referred to as balloon venoplasty). A balloon expandable stent may be placed over the balloon-tipped catheter so that when the balloon is expanded, the stent is imbedded into position. Alternatively, balloon venoplasty may be performed first, and a self-expanding stent delivered to the target over a guidewire and deployed. A repeat balloon dilation may then be performed over the newly imbedded stent.

Alternatively, a self-expanding stent is delivered to the target area first over a guide wire, deployed, and then dilated by a balloon inflation. Some prior art stent delivery systems for implanting self-expanding stents include an catheter having an inner lumen upon which the compressed or collapsed stent is mounted and an outer restraining sheath or tubular member that is placed over the compressed stent prior to deployment. The catheter is advanced to the target site over the guidewire, and the outer sheath is moved in relation to the inner lumen to “uncover” the compressed stent, allowing the stent to move to its expanded condition. Other types of deployment devices are known in the art.

Stents are generally cylindrically shaped devices which function to hold open and sometimes expand a segment of a blood vessel or other lumen. Stents may be balloon expandable or self expanding. The balloon expandable stent is a stent that is usually made of a coil, mesh or zigzag design and is constrained with a sheath. The stent is pre-mounted on a balloon and the inflation of the balloon plastically expands the stent with respect to the balloon diameter.

Self-expanding stents are a tubular device stored in an elongate configuration in what is called a delivery system or applicator. The applicator is introduced percutaneously into the body into a vessel at a suitable location, and guided through the vessel lumen to the location where the stent is to be released. The delivery system and the stent are often provided with radiological markers with which the positioning and the release of the stent can be monitored in situ under fluoroscopy. Upon release, the stent material auto expands to a predetermined size.

Commonly used self-expanding stents are braided stents, or laser cut stents. A braided stent is a metal stent that is produced by what is called a plain weaving technique. It is composed of a hollow body, which can stretch in the longitudinal direction and whose jacket is a braid made up of a multiplicity of filament-like elements which, in the expanded state of the braided stent, intersect a plane, perpendicular to the longitudinal direction, at a braid angle. A braided stent undergoes a considerable change in length when stretched (“foreshortening”), this change in length being all the greater the greater the original diameter and the smaller the original braid angle. (e.g. Wallstent from Boston Scientific (Boston, Mass. USA)). Because of the considerable shortening that takes place upon release of a braided stent, precise placement is difficult. However, braided stent advantages include their small delivery systems, higher resistance to external compression, and less propensity to fracture.

Laser cut stents are constructed from a tube of material (most frequently, nitinol (a nickel titanium alloy), and also stainless, cobalt, etc) that is laser-cut during production to create a meshed device. The tube is comprised of sequential aligned annular rings that are interconnected in a helical fashion. The tube is compressed and loaded into the delivery device and expands to original size when released. Nitinol, which has thermal memory, may help stents made of this material expand into position when exposed to body temperature after delivery. Compared with self-expanding braided stents, laser cut stents provide more accurate stent deployment with less foreshortening. An example of a commercially available laser cut stent is the SMART stent (Cordis, Fla. USA). Laser cut stents are much less subject to foreshortening and are probably less rigid than braided stents.

The stenting procedure is percutaneous, and the preferred stenting procedure is described in the inventors provisional application No. 61/151,075, entitled Venous System Stent Treatment, filed Feb. 9, 2009, hereby incorporated by reference. See also Neglen P, Berry M A, Raju S. Endovascular surgery in the treatment of chronic primary and post-thrombotic iliac vein obstruction. Eur J Vasc Endovasc Surg 2000; 20(6):560-71, and Neglen P, Raju S. Proximal lower extremity chronic venous outflow obstruction: recognition and treatment. Semin Vasc Surg 2002; 15(1):57-64, both hereby incorporated by reference.

Using this stenting technique to treat cellulitis, the inventors have found that long term (6 year) cumulative stent patency is remarkable at 100% in non-thrombotic disease and only slightly less in post-thrombotic disease. If the method were to fail, later open surgery to correct outflow obstruction or reflux is not precluded. Percutaneous laser ablation of the saphenous vein can be easily added to iliac vein stenting when indicated to maximize correction of pathology through minimally invasive techniques.

1. A method of treating recurrent cellulitis comprising the steps of (a) diagnosing the presence of cellulitis in the lower limbs; (b) determining that the cellulitis is recurrent; (c) examining the outflow veins for the presence of stenosis; (d) if stenosis representing a reduction of the expected cross-sectional area of the vein by about 50% or greater is found in a outflow vein area, stent the stenoic area.

2. The method of claim 1 wherein said examination of the outflow area is performed using IVUS.

3. The method of claim 1 wherein the ouflow veins comprise at least the iliac vein.

4. A method of treating chronic cellulitis comprising the steps of (a) diagnosing the presence of cellulitis in the lower limbs; (b) determining that the cellulitis is chronic; (c) examining the outflow veins for the presence of stenosis where the stenosis; (d) if stenosis representing a reduction of the expected cross-sectional area of the vein by about 50% or greater is found in a outflow vein area, stent the stenoic area.

5. The method of claim 4 wherein said examination of the outflow area is performed using IVUS.

6. The method of claim 4 wherein the ouflow veins comprise at least the iliac vein.

7. The method of claim 4 wherein said cellulitis is either spontaneous or non-spontaneous.

8. The method of claim 1 wherein said cellulitis is either spontaneous or non-spontaneous.

9. The method of claim 4 wherein said lower limbs consists of the legs.

10. The method of claim 1 wherein said lower limbs consists of the legs.
